(19) United States
(12) Patent Application Publication
(10) Pub. No.: US 2022/0409585 A1
(43) Pub. Date: Dec. 29, 2022

(54) TREATMENT OF AUTOINFLAMMATORY DISORDERS

(71) Applicant: Inflazome Limited, Dublin 2 (IE)

(72) Inventors: Matthew COOPER, Cambridge (GB); Luke Anthony John O'NEILL, Dublin 2 (IE)

(21) Appl. No.: 17/775,230

(22) PCT Filed: Nov. 6, 2020
(86) PCT No.: PCT/EP2020/081290

§ 371 Date: May 6, 2022

(30) Foreign Application Priority Data
Nov. 7, 2019 (GB) 1916238.7
Mar. 25, 2020 (GB) 2004335.2

(51) Int. Cl. A61K 31/415; A61P 29/00

(52) U.S. Cl. CPC A61K 31/415; A61P 29/00; C07B 2200/13; A61K 9/0053

(57) ABSTRACT

The present invention relates to a compound of formula (I) for use in the treatment or prevention of an autoinflammatory disorder such as cryopyrin-associated periodic syndrome (CAPS), tumor necrosis factor receptor associated periodic syndrome (TRAPS), hyperimmunoglobulin D syndrome (HIDS)/mevalonate kinase deficiency (MKD), familial mediterranean fever (FMF), Behcet Disease, Pyoderma Gangraenosum, systemic onset of juvenile idiopathic arthritis (sJIA), Schnitzler syndrome, or Hidradenitis Suppurativa.

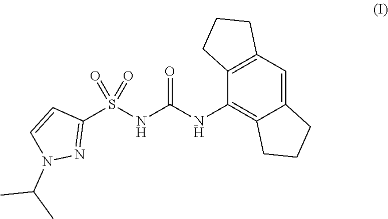 [SMILES: CC(C)n1ccc(S(=O)(=O)NC(=O)Nc2c3c(cc4c2CCC4)CCC3)n1]

The present invention relates to a compound of formula (I):

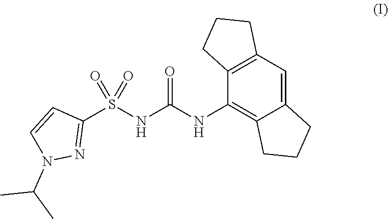 [SMILES: CC(C)n1ccc(S(=O)(=O)NC(=O)Nc2c3c(cc4c2CCC4)CCC3)n1]

for use in the treatment or prevention of an autoinflammatory disorder such as cryopyrin-associated periodic syndrome (CAPS), tumor necrosis factor receptor associated periodic syndrome (TRAPS), hyperimmunoglobulin D syndrome (HIDS)/mevalonate kinase deficiency (MKD), familial mediterranean fever (FMF), Behcet Disease, Pyoderma Gangraenosum, systemic onset of juvenile idiopathic arthritis (sJIA), Schnitzler syndrome, or Hidradenitis Suppurativa.

NLRP3 has been implicated in a number of autoinflammatory disorders, including tumor necrosis factor receptor associated periodic syndrome (TRAPS), hyperimmunoglobulin D syndrome (HIDS)/mevalonate kinase deficiency (MKD), and familial mediterranean fever (FMF) (Cook et al., Eur J Immunol, 40: 595-653, 2010; Timerman et al., J Clin Rheumatology, 19(8): 452-453, 2013; and Ozyilmaz et al., Int J Immunogenetics, 46: 232-240, 2019), Behcet Disease (Masters, Clin Immunol, 147(3): 223-228, 2013; Kim et al., J Inflammation, 12: article 41, 2015; and Yüksel et al., Int Immunol, 26(2): 71-81, 2014), Pyoderma Gangraenosum (Marzano et al., British Journal of Dermatology, 175(5): 882-891, 2016), systemic onset of juvenile idiopathic arthritis (sJIA) (Nirmala et al., Current Opinion in Rheumatology, 26(5): 543-552, 2014; Yang et al., Scandinavian Journal of Rheumatology, 43(2): 146-152, 2014; and Mejbri et al., Pediatric Drugs, 22: 251-262, 2020), Schnitzler syndrome (de Koning et al., J Allergy Clin Immunol, 135(2): 561-564, 2015; and Corcoran et al., Wellcome Open Research, 5:247, 2020), and Hidradenitis Suppurativa (Alikhan et al., J Am Acad Dermatol, 60(4): 539-61, 2009; Lima et al., British Journal of Dermatology, 174: 514-521, 2016; and Shah et al., Inflamm Res, 66: 931-945, 2017). In particular, NLRP3 mutations have been found to be responsible for a set of rare autoinflammatory diseases known as CAPS (Ozaki et al., J Inflammation Research, 8: 15-27, 2015; Schroder et al., Cell, 140: 821-832, 2010; and Menu et al., Clinical and Experimental Immunology, 166: 1-15, 2011). Cryopyrin-associated periodic syndromes (CAPS), also called cryopyrin-associated autoinflammatory syndromes, are three diseases related to a defect in the same gene: neonatal-onset multisystem inflammatory disease (NOMID), Muckle-Wells syndrome (MWS) and familial cold autoinflammatory syndrome (FCAS). The differences in these diseases lie in their severity and the organs involved. The aberrant activity of NLRP3 is pathogenic in CAPS. Although the other diseases mentioned above are not clinically diagnosed by mutations in the NLRP3 gene, the clinical phenotype including the periodic fever occurrence and their responsiveness to IL-1 inhibitors allow them to be categorized into the group of autoinflammatory diseases. There is also evidence showing that patients diagnosed with, for example, a condition that mimics FMF (Jeru et al., Arthritis & Rheumatism, 54(2): 508-514, 2006), Schnitzler syndrome (de Koning et al., J Allergy Clin Immunol, 135(2): 561-564, 2015) and Behcet Disease (Yuksel et al., Int Immunol, 26(2): 71-81, 2014), do harbour mutations in NLRP3 (https://infevers.umai-montpellier.fr/web/).

This invention is based on the discovery that the compound of formula (I) is particularly effective in the treatment of autoinflammatory disorders especially CAPS, most especially via the oral route.

In a first aspect of the present invention, there is provided a compound of formula (I):

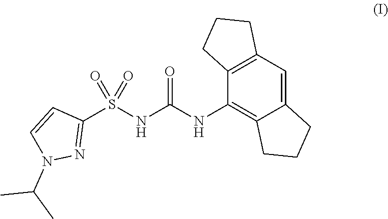 [SMILES: CC(C)n1ccc(S(=O)(=O)NC(=O)Nc2c3c(cc4c2CCC4)CCC3)n1]

or a pharmaceutically acceptable salt thereof, for use in the treatment or prevention of an autoinflammatory disorder.

In one embodiment, the autoinflammatory disorder is cryopyrin-associated periodic syndrome (CAPS). In one embodiment, the cryopyrin-associated periodic syndrome is Muckle-Wells syndrome (MWS). In another embodiment, the cryopyrin-associated periodic syndrome is familial cold autoinflammatory syndrome (FCAS). In another embodiment, the cryopyrin-associated periodic syndrome is neonatal-onset multisystem inflammatory disease (NOMID).

In one embodiment, the autoinflammatory disorder is tumor necrosis factor receptor associated periodic syndrome (TRAPS), hyperimmunoglobulin D syndrome (HIDS)/mevalonate kinase deficiency (MKD), familial mediterranean fever (FMF), Behcet Disease, Pyoderma Gangraenosum, systemic onset of juvenile idiopathic arthritis (sJIA), Schnitzler syndrome, or Hidradenitis Suppurativa.

In one embodiment, the treatment or prevention comprises the treatment or prevention of inflammation. Typically, the treatment or prevention of inflammation is achieved via NLRP3 inhibition. As used herein, the term “NLRP3 inhibition” refers to the complete or partial reduction in the level of activity of NLRP3 and includes, for example, the inhibition of active NLRP3 and/or the inhibition of activation of NLRP3.

In one embodiment, the treatment or prevention comprises the oral administration of the compound or the salt thereof. In a further embodiment, the treatment or prevention comprises the twice daily oral administration of the compound or the salt thereof. In a further embodiment, the treatment or prevention comprises the oral administration of the compound or the salt thereof at a dose of 2-4 mg/kg/dose, or at a dose of 3-3.6 mg/kg/dose, or at a dose of about 3.3 mg/kg/dose. In a further embodiment, the treatment or prevention comprises the twice daily oral administration of the compound or the salt thereof at a dose of 2-4 mg/kg/dose, or at a dose of 3-3.6 mg/kg/dose, or at a dose of about 3.3 mg/kg/dose.

In one embodiment, the compound or salt is a sodium salt, such as a monosodium salt. In one embodiment, the compound or salt is a monohydrate. In one embodiment, the compound or salt is crystalline. In one embodiment, the compound or salt is a crystalline monosodium monohydrate salt. In one embodiment, the crystalline monosodium monohydrate salt has an XRPD spectrum comprising peaks at: 4.3°2θ, 8.7°2θ, and 20.6°2θ, all ±0.2°2θ. In one embodiment, the crystalline monosodium monohydrate salt has an XRPD spectrum in which the 10 most intense peaks include 5 or more peaks which have a 2θ value selected from: 4.3°2θ, 6.2°2θ, 6.7°2θ, 7.3°2θ, 8.7°2θ, 9.0°2θ, 12.1°2θ, 15.8°2θ, 16.5°2θ, 18.0°2θ, 18.1°2θ, 20.6°2θ, 21.6°2θ, and 24.5°2θ, all ±0.2°2θ. The XRPD spectrum may be obtained as described in WO 2019/206871, which is incorporated in its entirety herein by reference.

In one embodiment, the crystalline monosodium monohydrate salt is as described in WO 2019/206871, which is incorporated in its entirety herein by reference. In one embodiment, the crystalline monosodium monohydrate salt has the polymorphic form described in WO 2019/206871, which is incorporated in its entirety herein by reference. In one embodiment, the crystalline monosodium monohydrate salt is prepared according to the method described in WO 2019/206871, which is incorporated in its entirety herein by reference.

Typically, in accordance with any embodiment of the first aspect of the invention, the treatment or prevention comprises the administration of the compound or the salt thereof to a patient. The patient may be any human or other animal. Typically, the patient is a mammal, more typically a human or a domesticated mammal such as a cow, pig, lamb, sheep, goat, horse, cat, dog, rabbit, mouse etc. Most typically, the patient is a human.

In a second aspect of the present invention, there is provided a pharmaceutical composition comprising a pharmaceutically acceptable excipient and a compound or salt of the first aspect of the present invention. In one embodiment, the pharmaceutical composition is suitable for oral administration.

In a third aspect of the present invention, there is provided a method for the treatment or prevention of an autoinflammatory disorder in a patient in need thereof, wherein the method comprises administering to the patient in need thereof a therapeutically or prophylactically effective amount of a compound of formula (I):

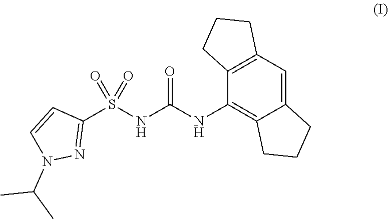 [SMILES: CC(C)n1ccc(S(=O)(=O)NC(=O)Nc2c3c(cc4c2CCC4)CCC3)n1]

or a pharmaceutically acceptable salt thereof.

In one embodiment, the autoinflammatory disorder is cryopyrin-associated periodic syndrome (CAPS). In one embodiment, the cryopyrin-associated periodic syndrome is Muckle-Wells syndrome (MWS). In another embodiment, the cryopyrin-associated periodic syndrome is familial cold autoinflammatory syndrome (FCAS). In another embodiment, the cryopyrin-associated periodic syndrome is neonatal-onset multisystem inflammatory disease (NOMID).

In one embodiment, the autoinflammatory disorder is tumor necrosis factor receptor associated periodic syndrome (TRAPS), hyperimmunoglobulin D syndrome (HIDS)/mevalonate kinase deficiency (MKD), familial mediterranean fever (FMF), Behcet Disease, Pyoderma Gangraenosum, systemic onset of juvenile idiopathic arthritis (sJIA), Schnitzler syndrome, or Hidradenitis Suppurativa.

In one embodiment, the treatment or prevention comprises the treatment or prevention of inflammation. Typically, the treatment or prevention of inflammation is achieved via NLRP3 inhibition.

In one embodiment, the treatment or prevention comprises the oral administration of the compound or the salt thereof. In a further embodiment, the treatment or prevention comprises the twice daily oral administration of the compound or the salt thereof. In a further embodiment, the treatment or prevention comprises the oral administration of the compound or the salt thereof at a dose of 2-4 mg/kg/dose, or at a dose of 3-3.6 mg/kg/dose, or at a dose of about 3.3 mg/kg/dose. In a further embodiment, the treatment or prevention comprises the twice daily oral administration of the compound or the salt thereof at a dose of 2-4 mg/kg/dose, or at a dose of 3-3.6 mg/kg/dose, or at a dose of about 3.3 mg/kg/dose.

In one embodiment, the compound or salt is a sodium salt, such as a monosodium salt. In one embodiment, the compound or salt is a monohydrate. In one embodiment, the compound or salt is crystalline. In one embodiment, the compound or salt is a crystalline monosodium monohydrate salt. In one embodiment, the crystalline monosodium monohydrate salt has an XRPD spectrum comprising peaks at: 4.3°2θ, 8.7°2θ, and 20.6°2θ, all ±0.2°2θ. In one embodiment, the crystalline monosodium monohydrate salt has an XRPD spectrum in which the 10 most intense peaks include 5 or more peaks which have a 2θ value selected from: 4.3°2θ, 6.2°2θ, 6.7°2θ, 7.3°2θ, 8.7°2θ, 9.0°2θ, 12.1°2θ, 15.8°2θ, 16.5°2θ, 18.0°2θ, 18.1°2θ, 20.6°2θ, 21.6°2θ, and 24.5°2θ, all ±0.2°2θ. The XRPD spectrum may be obtained as described in WO 2019/206871, which is incorporated in its entirety herein by reference.

In one embodiment, the crystalline monosodium monohydrate salt is as described in WO 2019/206871, which is incorporated in its entirety herein by reference. In one embodiment, the crystalline monosodium monohydrate salt has the polymorphic form described in WO 2019/206871, which is incorporated in its entirety herein by reference. In one embodiment, the crystalline monosodium monohydrate salt is prepared according to the method described in WO 2019/206871, which is incorporated in its entirety herein by reference.

In accordance with any embodiment of the third aspect of the invention, the patient may be any human or other animal. Typically, the patient is a mammal, more typically a human or a domesticated mammal such as a cow, pig, lamb, sheep, goat, horse, cat, dog, rabbit, mouse etc. Most typically, the patient is a human.

### EXPERIMENTAL

Figures

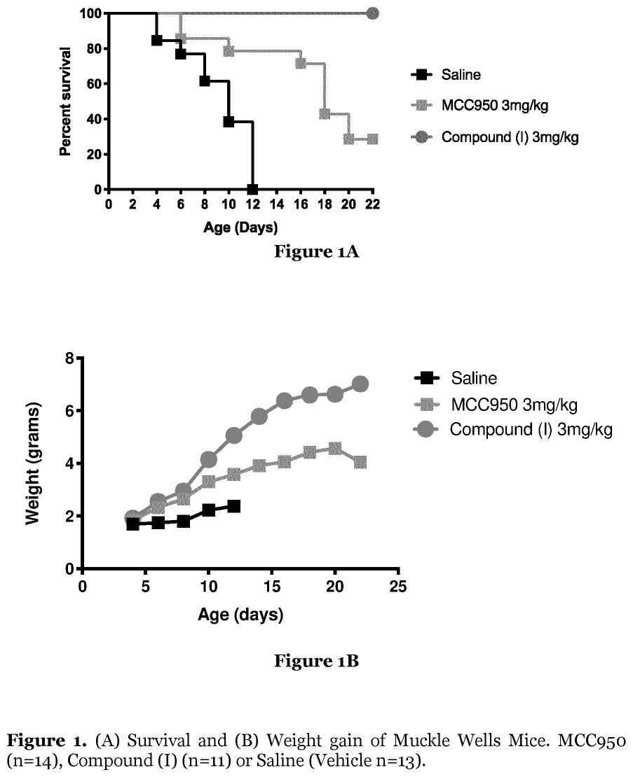

FIG. 1: FIG. 1A shows the survival rate and FIG. 1B the weight gain of the Muckle Wells mice of example 1.

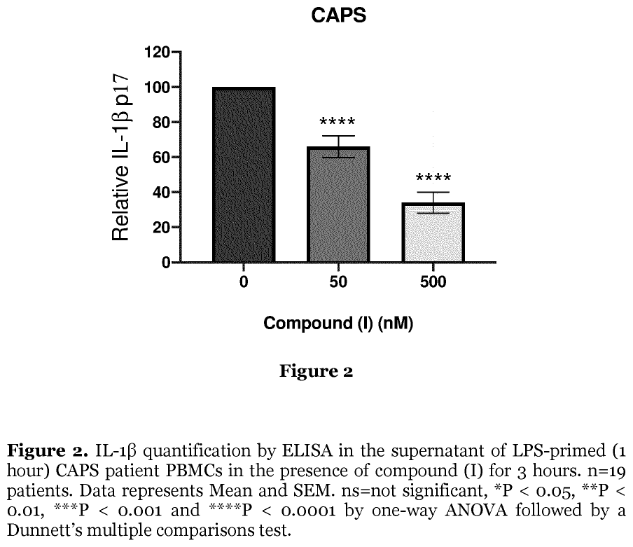

FIG. 2: IL-1β quantification by ELISA in the supernatant of LPS-primed (1 hour) CAPS patient PBMCs in the presence of compound (I) for 3 hours. n=19 patients. Data represents Mean and SEM. ns=not significant, *P<0.05, **P<0.01, ***P<0.001 and ****P<0.0001 by one-way ANOVA followed by a Dunnett's multiple comparisons test.

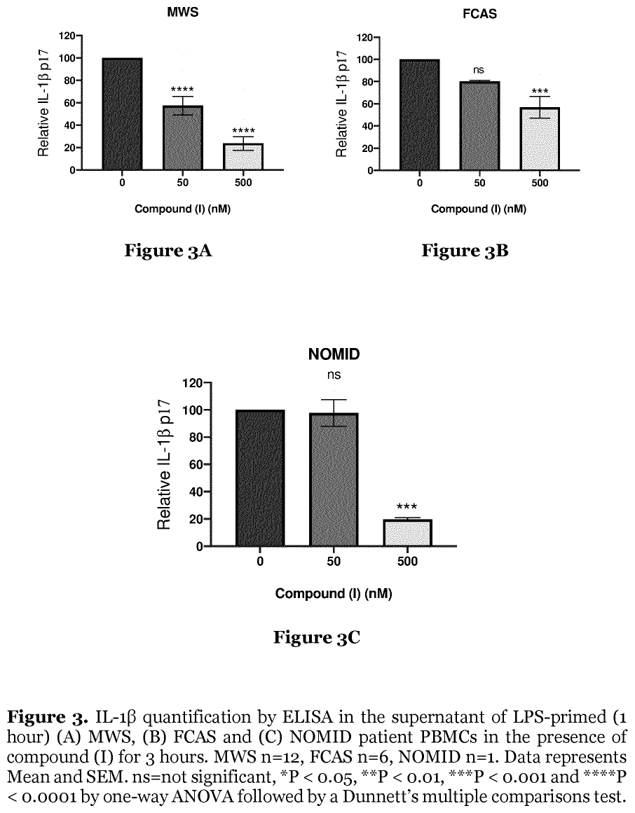

FIG. 3: IL-1β quantification by ELISA in the supernatant of LPS-primed (1 hour) (A) MWS, (B) FCAS and (C) NOMID patient PBMCs in the presence of compound (I) for 3 hours. MWS n=12, FCAS n=6, NOMID n=1. Data represents Mean and SEM. ns=not significant, *P<0.05, **P<0.01, ***P<0.001 and ****P<0.0001 by one-way ANOVA followed by a Dunnett's multiple comparisons test.

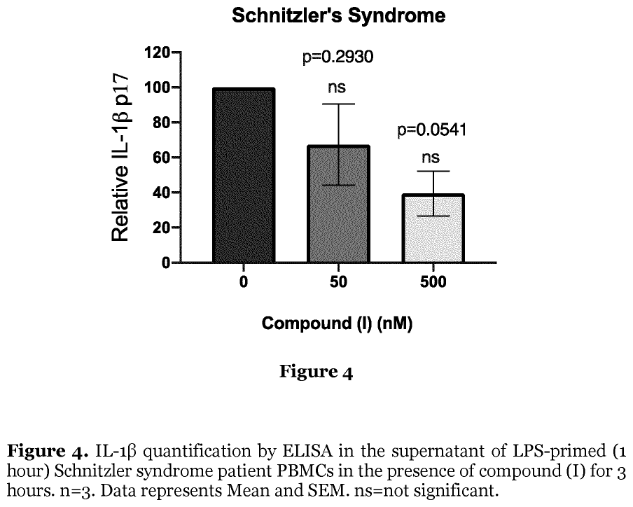

FIG. 4: IL-1β quantification by ELISA in the supernatant of LPS-primed (1 hour) Schnitzler syndrome patient PBMCs in the presence of compound (I) for 3 hours. n=3. Data represents Mean and SEM. ns=not significant.

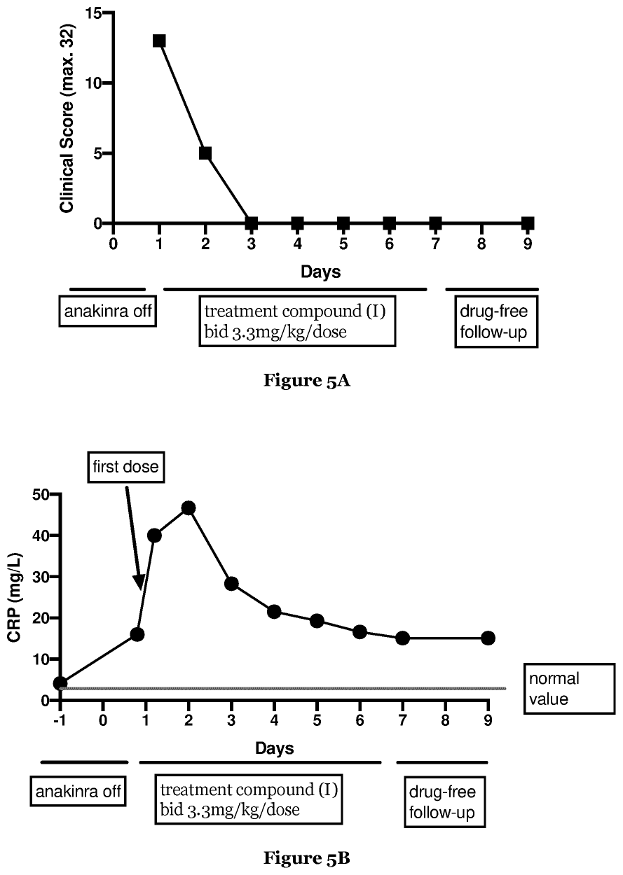

FIG. 5: FIG. 5A shows the clinical score results and FIG. 5B shows the C-reactive protein (CRP) results of example 3.

### EXAMPLE 1—MICE IN VIVO

### Material and Methods

### Animal Ethics

Ethical approval was obtained from the University of Queensland Animal Ethics Committee (ABS) prior to commencement of the study. All protocols conform to the NHMRC animal welfare guidelines.

### NWS Mouse Model and Intraperitoneal Injections

NLRP3-activating mutation in mice were backcrossed to C57BL/6 for at least ten generations. Heterozygote MWS-associated mutation Nlrp3 (A350VneoR) mice were crossed with homozygote LysMcre mice (B6.129P2-Lyz2^{tm1(cre)Ifo}/J). The NLRP3 mutant×LysM-Cre offspring were then injected with either saline (vehicle), MCC950 (3 mg/kg), or the compound of formula (I) (3 mg/kg) intraperitoneally every second day starting at day 4 after birth (P4). Where possible, a litter of saline-injected mice were included alongside each drug treatment experiment to ensure model consistency. The weight of each mouse was recorded daily, and dosing volumes adjusted accordingly, and mortalities or welfare euthanasias recorded. All mice still alive at day 22 were euthanised, and recorded as alive for the purpose of generating a survival curve.

### Results

In FIG. 1A, as expected, 100% of the control A350V mice (n=13) receiving no treatment (saline controls) succumbed to death within 12 days. Mice treated with a selective inhibitor of NLRP3, 3 mg/kg MCC950 (a commercially available tool compound used by the wider scientific community to study NLRP3 biology) (n=4), lead to improved survival of the A350V mice. In this instance ˜30% of the mice survived until day 22. However, treatment of neonatal mice with the compound of formula (I) at 3 mg/kg (n=ii) gave complete protection, with 100% of the animals surviving to termination of the study at day 22.

A general improvement of health was further indicated by the weight gain of the animals (FIG. 1B), mirroring the trend seen with the survival curves. The protection afforded by the compound of formula (I) led to this group steadily gaining the most weight over time.

The superiority of the compound of formula (I) in this model is further demonstrated over standard of care treatment, rilonacept. In a study published by Brydges et al. (Immunity, 30: 875-887, 2009), NlrP3 A350V/+/CreL mice treated with subcutaneously injected mouse form of rilonacept, mIL-1 Trap, every other day beginning day 1-2 post-birth, led to 100% of the animals succumbing to death by day ˜17 (Brydges et al., 2009). Despite doses up to 60 times those typically administered to humans, this led to only a three-day survival extension over control mice receiving no treatment (where all animals succumbed to death by day ˜14).

### EXAMPLE 2—HUMANS EX VIVO

Summary: The ex vivo activity of the compound of formula (I) on inhibition of cytokine release was determined in CAPS and Schnitzler syndrome patient PBMCs. Inhibition of IL-1β production by the compound of formula (I) was determined in 19 CAPS patient samples and 3 Schnitzler syndrome patient samples.

### Protocols

### Blood Collection

45 ml whole blood was collected from adult patients into Lithium Heparin tubes (Greiner VACUETTE® LH Lithium Heparin), and a volume appropriate to the patient age and weight from the paediatric patients. Following donation, blood samples were maintained at room temperature and PBMCs isolated from whole blood within 90 minutes of blood donation.

### PBMCs Isolation Procedure

1. Blood was decanted from collection tubes into 50 ml tubes and diluted 1:1 with PBS.
2. 20 ml diluted blood was layered over 15 ml of Lymphoprep™ (STEMCELL Technologies).
3. Samples were centrifuged for 20 mins at 400 g with brake OFF.
4. 5 ml of the PBS/serum layer was removed and stored at −80° C. for ELISA.
5. The majority of the remaining PBS/serum layer was removed and discarded, leaving around 2 ml PBS/serum.
6. The PBMC layer was removed using a pasteur pipette, and diluted to a volume of 50 ml with PBS.
7. The diluted PBMC layer was centrifuged for 10 mins at 300 g with brake ON.
8. The supernatant was discarded and the pellet was resuspended in 50 ml PBS.
9. The resuspended pellet was centrifuged for 10 min at 200 g with brake ON to remove platelets.
10. The pellet containing PBMC was resuspended in 10 ml media of serum free RPMI (+Pen/Strep).
11. Cells were counted. Normal harvest from 45 ml blood=30-80 million PBMCs.
12. Cells were seeded at 2×10⁶ cells/ml in 12-well plates in serum free RPMI (+Pen/Strep).
13. Cells were incubated for 2 hours at 37° C. before the start of the experiment.

### PBMC Assay for Protein Analysis by ELISA

1. 1 μg/mL of LPS was added into each well and cells were incubated for 1 hour at 37° C.
2. The medium was changed to serum free RPMI (+Pen/Strep).
3. The compound of formula (I) was added at 50 nM or 500 nM, and cells were incubated for 3 hours at 37° C.
4. The supernatant was harvested and stored at −80° C. until IL-1β quantification by ELISA.

### Measurement of IL-1β by ELISA Assays

At the termination of the experiments, the supernatants were collected for quantification of IL-1β by ELISA (Cat no. DLB50, R&D) as per manufacturer's standard procedure. Samples that were not analysed on the same day, were kept at −80° C.

### Results

Data on the compound of formula (I) are presented from ex vivo stimulated PBMCs. The data provide evidence that the compound of formula (I) can effectively block aberrant IL-1β production ex vivo in patients with active NLRP3-mediated disease. The compound of formula (I) acted to inhibit IL-1β production in MWS, FCAS and NOMID patient PBMC (FIGS. 2 and 3). The compound of formula (I) also showed a trend towards inhibition of IL-1β production in Schnitzler syndrome patient PBMC (FIG. 4).

### EXAMPLE 3—HUMANS IN VIVO

A 70 year old male CAPS patient was treated with compound (I). He was diagnosed with CAPS in 2010. His typical symptoms were rash, fever, fatigue, severe hearing loss, conjunctivitis, pain, and joint stiffness. He had been treated with anakinra since 2010 with almost normal C-reactive protein (CRP) since. He was found to have NLRP3 mutation Arg260Trp. He has three children and two grandchildren all affected by CAPS.

After cessation of anakinra and subsequent flaring, the patient was treated orally twice daily with 3.3 mg/kg/dose for 7 days. The treatment was well tolerated. The patient reinitiated anakinra at day 9.

Clinical score was recorded by daily physician assessment of skin disease, arthralgia, myalgia, headache/migraine, conjunctivitis, fatigue, and other symptoms, using a Likert scale (0=absent, 1=minimal, 2=mild, 3=moderate, 4=severe) summing up to a total of 32 points. It was found that clinical score improved within 2 days of treatment (FIG. 5A).

C-reactive protein (CRP) levels were measured. It was found that CRP levels reduced within 2 days of treatment (FIG. 5B).

1-18. (canceled)

19. A method for the treatment or prevention of an autoinflammatory disorder in a patient in need thereof, wherein the method comprises administering to the patient in need thereof a therapeutically or prophylactically effective amount of a compound of formula (I): 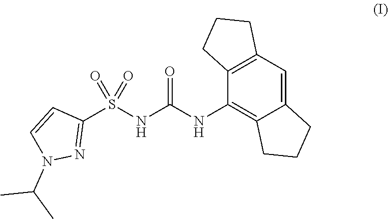 [SMILES: CC(C)n1ccc(S(=O)(=O)NC(=O)Nc2c3c(cc4c2CCC4)CCC3)n1]
or a pharmaceutically acceptable salt thereof.

20. The method as claimed in claim 19, wherein the autoinflammatory disorder is cryopyrin-associated periodic syndrome (CAPS).

21. The method as claimed in claim 20, wherein the cryopyrin-associated periodic syndrome is Muckle-Wells syndrome (MWS).

22. The method as claimed in claim 20, wherein the cryopyrin-associated periodic syndrome is familial cold autoinflammatory syndrome (FCAS).

23. The method as claimed in claim 20, wherein the cryopyrin-associated periodic syndrome is neonatal-onset multisystem inflammatory disease (NOMID).

24. The method as claimed in claim 19, wherein the autoinflammatory disorder is Schnitzler syndrome.

25. The method as claimed in claim 19, wherein the autoinflammatory disorder is tumor necrosis factor receptor associated periodic syndrome (TRAPS), hyperimmunoglobulin D syndrome (HIDS)/mevalonate kinase deficiency (MKD), familial mediterranean fever (FMF), Behcet Disease, Pyoderma Gangraenosum, systemic onset of juvenile idiopathic arthritis (sJIA), or Hidradenitis Suppurativa.

26. The method as claimed in claim 19, wherein the treatment or prevention comprises the treatment or prevention of inflammation.

27. The method as claimed in claim 19, wherein the treatment or prevention comprises the oral administration of the compound or the salt thereof.

28. The method as claimed in claim 19, wherein the compound or salt is a sodium salt.

29. The method as claimed in claim 1, wherein the compound or salt is a monosodium salt.

30. The method as claimed in claim 1, wherein the compound or salt is a monohydrate.

31. The method as claimed in claim 1, wherein the compound or salt is crystalline.

32. The method as claimed in claim 1, wherein the compound or salt is a crystalline monosodium monohydrate salt.

33. The method as claimed in claim 32, wherein the crystalline monosodium monohydrate salt has an XRPD spectrum comprising peaks at: 4.3°2θ, 8.7°2θ, and 20.6°2θ, all ±0.2°2θ.

34. The method as claimed in claim 32, wherein the crystalline monosodium monohydrate salt has an XRPD spectrum in which the 10 most intense peaks include 5 or more peaks which have a 2θ value selected from: 4.3°2θ, 6.2°2θ, 6.7°2θ, 7.3°2θ, 8.7°2θ, 9.0°2θ, 12.1°2θ, 15.8°2θ, 16.5°2θ, 18.0°2θ, 18.1°2θ, 20.6°2θ, 21.6°2θ, and 24.5°2θ, all ±0.2°2θ.

35. The method as claimed in claim 19, wherein the compound or the pharmaceutically acceptable salt thereof is administered as a pharmaceutical composition further comprising a pharmaceutically acceptable excipient.

36. The method as claimed in claim 35, wherein the pharmaceutical composition is suitable for oral administration.
